(19) United States
(12) Patent Application Publication
(10) Pub. No.: US 2022/0193008 A1
(43) Pub. Date: Jun. 23, 2022

(54) BIOACCESSIBILE COMPOSITIONS OF LIPOPHILIC COMPOUNDS AND PROCESS THEREOF

(71) Applicants: Shankaranarayanan JEYAKODI, Telangana Hyderabad (IN); Arunkanth KRISHNAKUMAR, Telangana Hyderabad (IN); Kalyan JANJANAM CHAKRAVARTHY, Telangana Hyderabad (IN)

(72) Inventors: Shankaranarayanan JEYAKODI, Telangana Hyderabad (IN); Arunkanth KRISHNAKUMAR, Telangana Hyderabad (IN); Kalyan JANJANAM CHAKRAVARTHY, Telangana Hyderabad (IN)

(21) Appl. No.: 17/603,955

(22) PCT Filed: Apr. 23, 2020
(86) PCT No.: PCT/IN2020/050379

§ 371 Date: Oct. 15, 2021

(30) Foreign Application Priority Data
Apr. 26, 2019 (IN) 201941016775

(51) Int. Cl. A61K 31/12; A61K 9/107; A61P 19/02; A61K 31/352; A61K 47/24; A61K 31/357; A61K 47/36; A61K 36/9066; A61K 36/324

(52) U.S. Cl. CPC A61K 31/12; A61K 9/1075; A61P 19/02; A61K 31/352; A61K 36/324; A61K 31/357; A61K 47/36; A61K 36/9066; A61K 47/24

(57) ABSTRACT

The present invention relates to a dispersible composition of lipophilic compounds. The present invention particularly relates to a dispersible composition for increasing the bioaccessibility of lipophilic compounds derived from plant and/or synthetic source. Further the present invention relates to a process for preparing the dispersible composition as an oral dosage form.

### CROSS REFERENCE TO RELATED APPLICATION

This application claims priority to co-pending Indian Provisional Patent Application Serial Number 201941016775 filed on Apr. 26, 2019. This application is incorporated herein by reference, in its entirety.

### TECHNICAL FIELD OF THE INVENTION

The present invention relates to a dispersible composition of lipophilic compounds. The present invention particularly relates to a dispersible composition for increasing the bioaccessibility of lipophilic compounds derived from plant and/or synthetic source. Further the present invention relates to a process for preparing the dispersible composition.

### BACKGROUND OF THE INVENTION

Phytochemical nutrients are natural compounds found in plants such as vegetables, fruits, whole grain products and legumes. These plant compounds have beneficial effects working with other essential nutrients to promote good health. Many phytochemical nutrients have antioxidant properties that help prevent damage to cells throughout the body. A number of phytochemical nutrients have been shown to reduce the risk of cancer, heart disease, stroke, Alzheimer's and Parkinson's disease.

Phytochemical nutrients such as curcumin, tocopherols, tocotrienols, carotenoids, plant sterols and stanols, and lecithins, select omega-3 fatty acids and polyunsaturated fatty acids have lipophilic characteristic. Lipophilic nutrients, by nature, are water insoluble products. Due to their insolubility their bioavailability is very poor. Lipophilic nutrients have limited absorption in the body due to limited solubility in gastrointestinal tract.

Curcumin is the major bioactive component of the spice herb turmeric or Curcuma longa L., a widely used natural food product in curry powder. Curcumin is limited by its poor solubility, low absorption from the gut, rapid metabolism and rapid systemic elimination. While the major portion of ingested curcumin is excreted through the faeces unmetabolized, the small portion absorbed is extensively converted to its water-soluble metabolites, glucuronides and sulfates. Curcumin is biotransformed, predominantly in the liver, to dihydrocurcumin and tetrahydrocurcumin and these metabolites are converted to mono-glucuronidated conjugates. Curcumin-, dihydrocurcumin-, and tetrahydrocurcumin glucuronides, as well as tetrahydrocurcumin (THC) are the major metabolites of curcumin in vivo (Pan, M. H., Huang, T. M., Lin, J. K., Biotransformation of curcumin through reduction and glucuronidation in mice. Drug Metab. Dispos. 1999, 27, 486-494). As Curcumin is insoluble in water, the in-vivo absorption and bioavailability is subsequently very minimal. Hence large amounts of Curcumin must be taken orally, though it reaches a maximum of only 2% absorption (Ralf Jager. Comparative absorption of curcumin formulations. Nutr J. 2014; 13:11)

By nature lipophilic nutrients are unstable in presence of oxygen and light. Therefore, they can be stabilized by the incorporation of certain stabilizing antioxidants. To further enhance stability, the lipophilic nutrients can be coated with polymer(s) which provide protection against the harmful effects of oxygen, light and moisture. Many nutritional formulations in the industry are in the form of tablets, capsules or dry-mixes. It is a major problem for formulators and manufacturers of such supplements to incorporate lipophilic nutrients such as curcumin, carotenoids, vitamin E sources like tocopherols and tocotrienols, concentrated forms of PC-rich lecithins, phytosterols, etc into dry forms due to the oily, waxy or viscous nature of these products.

The reasons for reduced bioavailability of any agent within the body may be attributed to low intrinsic activity, poor absorption, high rate of metabolism, inactivity of metabolic products and/or rapid elimination and clearance from the body. Studies on curcumin relating to absorption, distribution, metabolism and excretion of curcumin have revealed poor absorption and rapid metabolism of curcumin severely curtails its bioavailability.

W02007103435 discloses curcuminoid formulations having enhanced bioavailability comprising of a curcuminoid, antioxidant, glucuronidation inhibitor and water-soluble pharmaceutically acceptable inhibitor which are useful for treating Alzheimer's disease and other age-related disorders.

Indian patent application 1827/DEL/2008 provides for curcumin nanoparticles and curcumin bound to chitosan nanoparticles and methods of producing the same. The bioavailability of curcumin in these formulations was shown to improve by more than 10 fold.

WO2012156979 describes a water soluble composition having enhanced bioavailability useful for the treatment of depression which comprises a synergistic combination of curcumin, at least an antioxidant, a hydrophilic carrier and a fat.

WO2014094921 discloses a solubilizate consisting of curcumin and at least one emulsifier having an HLB value in the range of 13 and 18.

U.S. Ser. No. 10/022,416B2 provides a highly bioavailable, soluble, sustained release nano formulation comprising a hydrophobic plant derived compound(s) in an emulsifier phase, and aqueous phase.

Evidence from numerous literatures revealed that curcumin has poor absorption, bio distribution, metabolism, and bioavailability. Thus, continuous research on curcumin found some possible ways to overcome these problems.

### OBJECT OF THE INVENTION

An object of the present invention is to provide a dispersible composition of lipophilic compounds.

Another object of the present invention is to provide a dispersible composition of lipophilic compounds, derived from plant and/or synthetic source, having enhanced bioaccessibility.

Yet another object of the present invention is to provide a dispersible composition of lipophilic active(s) like curcumin, demethoxycurcumin, bisdemethoxycurcumin, bis-o-demethylcurcumin, tetrahydro curcumin, lutein, zeaxanthin, carotenoids, beta-carotene, boswellic acid, green tea extract, green coffee extract, resveratrol, hypericin, bacosides, xantho compounds/extracts, rhizol, pyrogallol, genistein, wogonin, morin, silymarin, flavonols like quercetin, froskolin and kaempferol; flavones like luteolin and apigenin; hydroxybenzoic acid like gallic acid, protocatechuic acid, ellagic acid (EA), and vanillic acid; flavanones cardinal aglycones like naringenin, hesperetin, and eriodictyol.

Another object of the present invention is to provide an oral dosage form of the dispersible composition of lipophilic active(s).

Another object of the present invention is to provide a dispersible composition comprising lipophilic active(s), hydrophilic carrier, micelle forming agent and optionally an acidifier.

Another object of the present invention is to provide a dispersible composition of curcumin extract that results in product containing bioaccessible curcumin (concentration achieved is 5%, 10%, 20%, 40% and 50%).

Yet another object of the present invention is to provide bioaccessible curcumin that shows therapeutic effect at a minimal daily intake of 500 mg, which is equivalent to 100 mg of Curcuminoids.

Another object of the present invention is to provide bioaccessible curcumin that can be used as an anti-inflammatory, anticancer, antimicrobial, neuroprotective and also significantly for the prophylaxis of rheumatoid arthritis, osteoarthritis, liver cirrhosis and asthma.

Another object of the present invention is to provide bioaccessible curcumin that helps in elevating testosterone level, promotes weight loss by lipolysis and has potential antioxidant effect.

Still another object of the present invention is to provide a process for preparing the dispersible composition of lipophilic compounds.

### SUMMARY OF THE INVENTION

An embodiment of the present invention is to provide a dispersible composition of lipophilic compounds.

Another embodiment of the present invention is to provide a dispersible composition of lipophilic compounds, derived from plant and/or synthetic source, having enhanced bioaccessibility.

Yet another embodiment of the present invention is to provide a dispersible composition of lipophilic active(s) like curcumin, demethoxycurcumin, bisdemethoxycurcumin, bis-o-demethylcurcumin, tetrahydro Curcumin, lutein, zeaxanthin, carotenoids, beta-carotene, boswellic acid, green tea extract, green coffee extract, resveratrol, hypericin, bacosides, xantho compounds/extracts, rhizol, pyrogallol, genistein, wogonin, morin, Silymarin, flavonols like quercetin, froskolin and kaempferol; flavones like luteolin and apigenin; hydroxybenzoic acid like gallic acid, protocatechuic acid, ellagic acid (EA), and vanillic acid; flavanones cardinal aglycones like naringenin, hesperetin, and eriodictyol.

An embodiment of the present invention is to provide a dispersible composition of curcumin having enhanced bioaccessibility.

Another embodiment of the present invention is to dispense the dispersible composition as an oral dosage form.

Yet another embodiment of the present invention is to provide a dispersible composition comprising lipophilic active(s), hydrophilic carrier, micelle forming agent and optionally an acidifier.

Another embodiment of the present invention is to provide a dispersible composition of curcumin extract that results in product containing dispersible curcumin (5-50%). Preferably the resulting dispersible curcumin concentration achieved is 5%, 10%, 20%, 40% and 50%.

Yet another embodiment of the present invention is to provide curcumin that shows therapeutic effect at a minimal daily intake of 500 mg, which is equivalent to 100 mg of Curcuminoids.

Another embodiment of the present invention is to provide bioaccessible curcumin that can be used as an anti-inflammatory, anticancer, antimicrobial, neuroprotective and also and also significantly for the prophylaxis of rheumatoid arthritis, osteoarthritis, liver cirrhosis and asthma.

Still another embodiment of the present invention is to provide bioaccessible curcumin that helps in elevating testosterone level, promotes weight loss by lipolysis and has potential antioxidant effect.

Another embodiment of the present invention is to provide a process for preparing the dispersible composition of lipophilic compounds.

### BRIEF DESCRIPTION OF DRAWINGS

In order to better understand the subject matter that is disclosed herein and to exemplify how it may be carried out in practice, embodiments will now be described, by way of non-limiting example only, with reference to the accompanying drawings, in which:

FIG. 1 presents effect of Bioaccessible curcumin (obtained from present invention) and Curcumin extract on solubility measured at regular time intervals

FIG. 2 presents effect of Bioaccessible curcumin (obtained from present invention) and Curcumin extract on solubility rate

FIG. 3 presents effect of Bioaccessible curcumin (obtained from present invention) and Curcumin extract in in-vitro dissolution

FIG. 4 presents bioavailability comparison between Bioaccessible curcumin and curcumin extract in adult rats

FIG. 5 presents maximum plasma concentration comparison between Bioaccessible curcumin and curcumin extract in adult rats

FIG. 6 presents WOMAC Pain Reduction comparative data for Bioaccessible curcumin and placebo

FIG. 7 presents WOMAC Stiffness Reduction comparative data for Bioaccessible curcumin and placebo

FIG. 8 presents WOMAC Physical Function comparative data for Bioaccessible curcumin and placebo

FIG. 9 presents WOMAC Index comparative data for Bioaccessible curcumin and placebo

FIG. 10 presents Percent improvement in VAS Pain Score in case of Rheumatoid arthritis patients consuming bioaccessible curcumin capsules.

### DETAILED DESCRIPTION OF THE INVENTION

In order to make the matter of the invention clear and concise, the following definitions are provided for specific terms used in the following description.

This term “Bioaccessibility” used in the present specification refers to the fraction of the total amount of a substance that is potentially available for absorption.

The term “Bioavailability” used throughout the specification means one of the principal pharmacokinetic properties of any compound that may be used as drug/active ingredient. It is the measure of presence of drug/active ingredient in the systemic circulation once taken orally in terms of solubility, dispersibility etc.

The term “Solubility” means the property of any compound to dissolve in a solid, liquid, or gas to form a homogeneous solution.

In an embodiment of the present invention there is provided a dispersible composition of lipophilic nutrients. Preferably the present invention provides a dispersible composition of lipophilic nutrients, derived from plant and/or synthetic source, having enhanced bioaccessibility.

The lipophilic active(s) used in the present invention maybe selected from curcumin, demethoxycurcumin, bisdemethoxycurcumin, bis-o-demethylcurcumin, tetrahydro Curcumin, lutein, zeaxanthin, carotenoids, beta-carotene, boswellic acid, green tea extract, green coffee extract, resveratrol, hypericin, bacosides, xantho compounds/extracts, rhizol, pyrogallol, genistein, wogonin, morin, Silymarin, flavonols like quercetin, froskolin and kaempferol; flavones like luteolin and apigenin; hydroxybenzoic acid like gallic acid, protocatechuic acid, ellagic acid (EA), and vanillic acid; flavanones cardinal aglycones like naringenin, hesperetin, and eriodictyol.

In an embodiment the lipophilic active used in the present invention is preferably curcumin. The term “Curcumin” means the principal curcuminoids found in turmeric, which is a member of the ginger family.

In further embodiment, when curcumin extract is used as lipophilic active in the process of present invention the resulting product is dispersible curcumin (5-50%). The preferable dispersible curcumin concentration achieved is 5%, 10%, 20%, 40% and 50%.

The dispersible curcumin uniquely improves the bioaccessibility over the curcumin developed by any prior art processes. The dispersible curcumin obtained by process of present invention prevents the efflux through P-glycoprotein, modulates the cytochrome P450 enzymes, enhances the permeation mechanism and significantly increases solubility thus resulting in enhanced bioaccessibility.

The test conducted to prove enhanced bioaccessibility shows that P-glycoprotein (P-gp), is an energy-dependent transporter protein located in the apical membrane of intestinal mucosal cells. Literature survey reveals that efflux transporter such as P-gp, BCRP and MRP may limit the bioavailability of many orally administered drugs¹. The mechanism involved is transportation of drug molecule back into the intestinal lumen after their absorption by the enterocytes. These inhibitors may play a significant role in the enhancement of oral absorption of the drugs with poor bioavailability due to the activity of mucosal P-gp.

Reference: L. Barthe, M. Bessouet, J. F. Woodley, G. Houin, The improved everted gut sac: a simple method to study intestinal P-glycoprotein, Inter Journal of Pharmaceutics 173 (1998) 255-258.

The permeability study conducted as Caco-2 Permeability Study, shows that the Apparent permeability of bioaccessible Curcumin powder of the present invention was found to be 0.021×10-06 cm/sec. Whereas the apparent permeability of Curcumin powder was found to be 0.442×10-06 cm/sec. These results indicate that p-gp inhibition of bioaccessible Curcumin powder of the present invention is greater than Curcumin powder. This directly indicates that permeability of bioaccessible Curcumin powder of the present invention in Caco-2 cells is higher compared to Curcumin powder. The bioaccessible Curcumin powder of the present invention prevents efflux mechanism thus increases the permeation in cells.

The dispersible composition of the present invention comprises lipophilic active(s), hydrophilic carrier, micelle forming agent and optionally an acidifier.

The present invention provides a dispersible composition of lipophilic compounds by forming micellar structure. Micelles are formed by self-assembly of amphiphilic molecules. Micelle can be used to increase the solubility of material that are normally insoluble or poorly soluble in dispersed medium. Micellization protects drug molecules from degradation via hydrolysis or other physicochemical reactions. This increases their shelf life, or prolongs their stability during use. Further micellization increases the bioaccessibility, lengthens drug circulation, enhances permeability and reduces rate of metabolism.

In the composition of present invention the range of different ingredients is as follows:

Lipophilic active—1 to 60% preferably 5-50%

Hydrophilic carrier—10-90% preferably 40-90%

Micelle forming agent—1-50% preferably 1-5%

Acidifier (optionally)—0.5-10% preferably 1-5%

The hydrophilic carrier used in the present invention maybe selected from Carboxymethyl cellulose, methyl cellulose, hydroxyethyl cellulose, Hydroxypropyl cellulose, Hydroxypropyl methyl cellulose, starch, modified starch, gelatin, lactose, mannitol, acacia, carbomer, dextrin, xanthan gum, Arabic gum, maltodextrin, aqueous shellac, liquid glucose, polyvinyl pyrrolidone, polyethylene glycol, glycerol, sucrose and the like, and combinations thereof.

The micelle forming agent used in the composition of the present invention maybe selected from vegetable or edible oils, polysorbates, lecithins, sucrose ester gums, penova ester gums, phopsphotidylcholines, glycerol and derivatives, glyceryl mono stearates, glyceryl distearate, etc.

The acidifier, if used in the composition of the present invention, maybe selected from citric acid, ascorbic acid, tartaric acid, malic acid, fumaric acid, lactic acid, formic acid, acetic acid, propionic acid, butyric acid, sorbic acid, etc. or combination thereof depending on the end formulation.

In another embodiment of the present invention the dispersible composition of lipophilic compounds having enhanced bioaccessibility is prepared by a process comprising of—
(a) dissolving the lipophilic active(s) in a solvent or mixture of solvents to form a clear solution
(b) dissolving the hydrophilic carrier in water
(c) adding the lipophilic active phase in hydrophilic carrier phase under homogenization to form a micelle
(d) dispersing the micelle in a micelle forming agent optionally with an acidifier
(e) removing the solvent and drying to obtain powder.

The present invention discloses bioaccessible composition of lipophilic actives wherein the lipophilic active is encapsulated in submicron size micelles formed by combination of hydrophilic polymer and emulsifiers. The choice of hydrophilic polymer preferably emulsifying modified starch based polysaccharides. This process based on encapsulation with emulsifying modified starch in submicron size range with micellar formation using lipids. This product is totally unique in terms of encapsulation matrix and process used for improving bioaccessibility of lipophilic actives.

The solvent used in the process maybe selected from isopropyl alcohol, methylene dichloride, ethyl alcohol, ethyl acetate and the like.

The average diameter of the micelles formed in the process of the present invention depends on the particle size of the lipophilic active.

In an embodiment, the dried product obtained from the process of the present invention maybe formulated as powder and granules which can be used in formulation of tablet, capsule, powder mixes or ready to drink beverages.

The bioaccessible curcumin, obtained from the composition of the present invention, can be used as an anti-inflammatory, anticancer, antimicrobial, neuroprotective and also significantly for the prophylaxis of rheumatoid arthritis, arthritis, liver cirrhosis, asthma, osteoarthritis. It acts as a potential antioxidant, helps in elevating testosterone level, promotes weight loss by lipolysis.

In an embodiment the bioaccessible curcumin obtained by the process of the present invention shows therapeutic effect at a minimal daily intake of 500 mg, which is equivalent to 100 mg of Curcuminoids. With a minimal daily dose of 500 mg bioaccessible curcumin, the investigational product showed better efficacy with no adverse effects. It is important to notice that very commonly recommended daily dose of Curcumin starts with 1-2 g and based on the clinical outcome (provided in the example section of the specification) bioaccessible curcumin stands unique as a potent therapeutic/prophylactic Ingredient.

Also the clinical study outcome provided in the example section of this specification, helps in concluding that bioaccessible curcumin, obtained from the disclosed invention, is a safe and well tolerated effective treatment for reducing symptoms of rheumatoid arthritis along with reducing associated pain and stiffness. Further, the bioaccessible curcumin of present invention helps to improve the physical function for healthy middle-aged and older RA patients.

In an embodiment the stability testing of the bioaccessible curcumin obtained by present invention shows that it is highly stable under recommended storage conditions. The Bioaccessible curcumin, obtained by the present invention, was taken and packed in aluminium pouch (accelerated condition 40°±2° & 75%±5% RH). The sample timings were Day 0, 3 months and on completion of 6 months. The results of the stability test are:

| | | LOD | | |
|---|---|---|---|---|
| Sampling | | (at | Assay % | |
| time | Description | 105° C.) | (Curcuminoid) | Dispersibility |
| Initial | Yellowish | 5.5 | 22.70% | Yellow colored |
| | orange powder | | | dispersion in |
| | | | | water |
| 3 months | Yellowish | 6.1 | 21.80% | Yellow colored |
| | orange powder | | | dispersion in |
| | | | | water |
| 6 months | Yellowish | 6.2 | 22.61% | Yellow colored |
| | orange powder | | | dispersion in |
| | | | | water |

The Bioaccessible curcumin, obtained by the present invention, was administered to adult rats to understand the bioavailability and plasma concentration of Curcuminoids. Curcumin powder was used as comparator for the study. The study results proved that with the bioaccessible curcumin composition, of the present invention, there was increase in bioavailability of Curcuminoids. This justified that the novel composition and process of the present invention attributed to enhanced solubility and bioavailability of Bioaccessible curcumin.

The nature of the invention, its objects and advantages are explained hereunder in greater detail in relation to non-limiting exemplary embodiments.

The following examples are for the purpose of illustration of the invention only and are not intended in any way to limit the scope of the present invention.

### EXAMPLES

### Example 1

| Sr. No | Name of Ingredient | Qty for 100 g | |
|---|---|---|---|
| 1 | Boswellia Extract | 23 | g |
| 2 | Methylene Dichloride | 360 | g |
| 3 | Modified Starch | 65 | g |
| 4 | Lecithin | 6 | g |
| 5 | Glyceryl monostearate | 6 | g |
| 6 | Purified Water | 200 | g |

23 g of Boswellia extract, 6 g of lecithin and 6 g of glyceryl monostearate were dissolved in 360 g of methylene dichloride. Separately 200 g purified water was heated at 50° C. and in it 65 g of modified starch was dissolved. The solvent phase was added slowly to the aqueous phase using high shear force mixer. After the addition was complete, the emulsion temperature was maintained at 40° C. while high speed shear mixing continued for 30 min. The temperature was gradually raised and mixing was continued until all solvent evaporated. Distilled water was added and contents were thoroughly mixed. The emulsion obtained was dried using rotary evaporator. The solid material obtained was further milled to get uniform powder.

### Example 2

| Sr. No | Name of Ingredient | Qty for 100 g | |
|---|---|---|---|
| 1 | Curcumin Extract | 6 | g |
| 2 | Acetone | 50 | g |
| 3 | Polyethylene glycol 6000 | 20 | g |
| 4 | Maltodextrin | 66 | g |
| 5 | Lecithin | 6 | g |
| 6 | Colloidal silicon dioxide | 2 | g |

20 g of Polyethylene glycol 6000 was melted at 75° C. and in it 6 g curcumin extract and 6 g lecithin were dissolved. The solution was cooled and in it 50 g acetone was added. To this 66 g of maltodextrin was added. The temperature was gradually raised and mixing continued till all solvent got evaporated. The solidified material obtained was further milled to get uniform dry powder.

### Example 3

| Sr. No | Name of Ingredient | Qty for 100 g | |
|---|---|---|---|
| 1 | Curcumin Extract | 23 | g |
| 2 | Acetone | 230 | g |
| 3 | Methylene Dichloride | 460 | g |
| 4 | Modified Starch | 70 | g |
| 5 | Guar gum | 5 | g |
| 6 | Citric acid | 2 | g |
| 7 | Purified Water | 200 | g |

23 g of curcumin extract was dissolved in mixture of 230 g acetone and 460 g methylene dichloride. 70 g modified starch, 5 g guar gum and 2 g citric acid were dissolved in 200 g of heated purified water. The solvent phase was added slowly to the aqueous phase using high shear force mixer. After the addition was complete, the emulsion was continued to mix at 50° C. for 15 min. The temperature was gradually raised and mixing was continued until all solvent got evaporated. To the resulting emulsion, distilled water was added and contents were thoroughly mixed. The contents were then sprayed using spray dryer to obtain uniform powder.

### Example 4

| Sr. No | Name of Ingredient | Qty for 100 g | |
|---|---|---|---|
| 1 | Curcumin Extract | 46 | g |
| 2 | Ethanol | 460 | g |
| 3 | Methylene Dichloride | 750 | g |
| 4 | Modified Starch | 46 | g |
| 5 | Glyceryl mono stearate | 6 | g |
| 6 | Citric acid | 2 | g |
| 7 | Purified Water | 150 | g |

46 g of curcumin extract was dissolved in mixture of 460 g ethanol and 750 g methylene dichloride. 46 g modified starch, 6 g of glyceryl monostearate and 2 g of citric acid were dissolved in 200 g of heated purified water. The solvent phase was added slowly to the aqueous phase using high shear force mixer. After the addition was complete, the emulsion was continued to mix at 50° C. for 15 min. The temperature was gradually raised and mixing was continued until all solvent got evaporated. To the resulting emulsion, distilled water was added and contents were thoroughly mixed. The contents were then sprayed using spray dryer to obtain uniform powder.

### Example 5

| Sr. No | Name of Ingredient | Qty for 100 g | |
|---|---|---|---|
| 1 | Froskolin Extract | 6 | g |
| 2 | Isopropyl alcohol | 30 | g |
| 3 | Polyethylene glycol 4000 | 30 | g |
| 4 | Maltodextrin | 53 | g |
| 5 | Lecithin | 6 | g |
| 6 | Colloidal silicon dioxide | 5 | g |

30 g of Polyethylene glycol 4000 was melted at 55° C. and to it 6 g of froskolin extract and 6 g of lecithin were added. The solution was cooled up to 35° C. and 30 g isopropyl alcohol was added. In this 53 g of maltodextrin was added and contents were mixed using rapid mixer granulator at high shear. The temperature was gradually raised and all solvent was allowed to evaporate. The solid material obtained was milled to get uniform dry powder.

### Example 6

| Sr. No | Name of Ingredient | Qty for 100 g | |
|---|---|---|---|
| 1 | Silymarin extract | 46 | g |
| 2 | Ethanol | 300 | g |
| 3 | Methylene Dichloride | 500 | g |
| 4 | Modified Starch | 46 | g |
| 5 | Phospholipids | 10 | g |
| 6 | Purified Water | 150 | g |

46 g of silymarin extract and 10 g phospholipid were dissolved in a mixture of 300 g ethanol and 500 g methylene dichloride. 200 g of purified water was heated at 50° C. and 46 g modified starch was dissolved in it. The solvent phase was added slowly to the aqueous phase using a high shear force mixer. After the addition was complete, the emulsion temperature was maintained at 50° C. and mixing was continued for 45 min. The temperature was gradually raised and mixing was continued until all solvent got evaporated. To this emulsion, distilled water was added and contents were thoroughly mixed. The contents were then sprayed using spray dryer and uniform powder was obtained.

### Example 7: Comparative Solubility Study Between Bioaccessible Curcumin, Obtained from Example 3, and Curcumin Extract

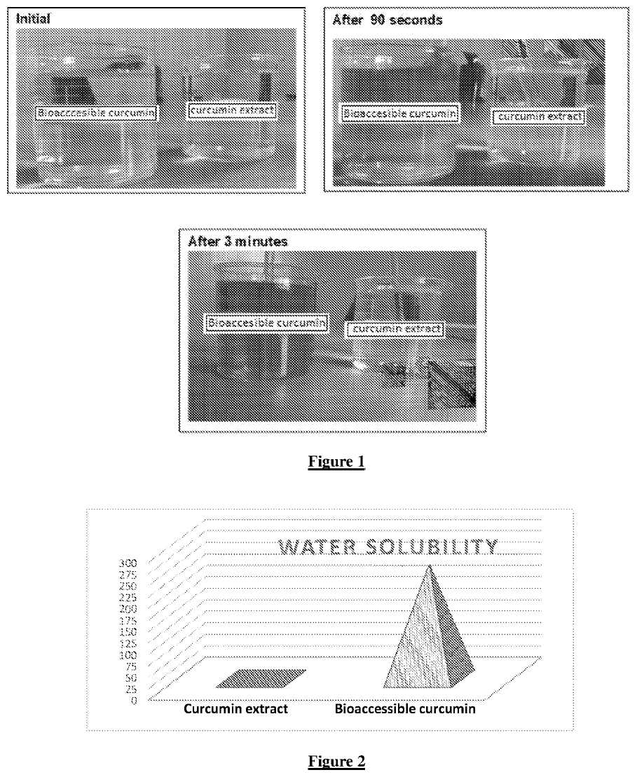

The solubility of bioaccessible Curcumin (developed by present invention) and Curcumin extract (starting material of the present invention) was compared by manually stirring both the ingredients separately in 240 mL cold water under similar conditions. The solubility level of both the materials was monitored at regular intervals i.e. initial, 90 seconds and 3 minutes. The results are shown in FIG. 1. The solubility rate was plotted on a graph as shown in FIG. 2. The results exhibited that bioaccessible Curcumin has superior water solubility and significantly rapid dissolution than Curcumin extract.

### Example 8: In-Vitro Dissolution Study Comparison Between Bioaccessible Curcumin, Obtained from Example 3, and Curcumin Extract

Dissolution studies were carried out in simulated gastric fluid, which mimics the in-vivo gastric conditions and directly correlates with the in-vivo bioavailability. The USP Type II dissolution apparatus (paddles) was used. 900 ml of simulated gastric fluid with 2.0% sodium lauryl sulfate was used as medium. The paddles were rotated at 75 rpm and temperature was maintained at 37±0.5° C. The dissolution profile of 500 mg bioaccessible curcumin in a capsule was compared with the dissolution of 500 mg Curcumin extract in a separate capsule. The dissolution profile was plotted on a graph as shown in FIG. 3.

### Example 9: Comparative Bioavailability Study

The bioavailability and plasma concentration of Curcuminoids in adult rats after administration of Curcumin 95% Powder and Bioaccessible curcumin (obtained from any Example 1-6) was studied. The study was carried out in two Groups, each with 6 rats, total of 12 Male Wistar rats and the dose administered was 500 mg/kg at given single dose for both Curcumin 95% powder and Bioaccessible curcumin of present invention. Blood withdrawn at 0.00, 0.50, 1.00, 2.00, 4.00, 6.00, 8.00, 12.00- and 24.00-hour time points from retro orbital vein using heparin as anticoagulant.

The study was majorly focused on evaluating absorption and plasma concentration by measuring:

a) plasma concentration of Curcuminoids to be measured at different time points by HPLC.

b) Cmax—The maximum plasma concentration of the active ingredient

c) AUC—The amount of active ingredient that is present in the circulation in a determined time period.

Curcuminoids levels were measured and compared in both group of animals.

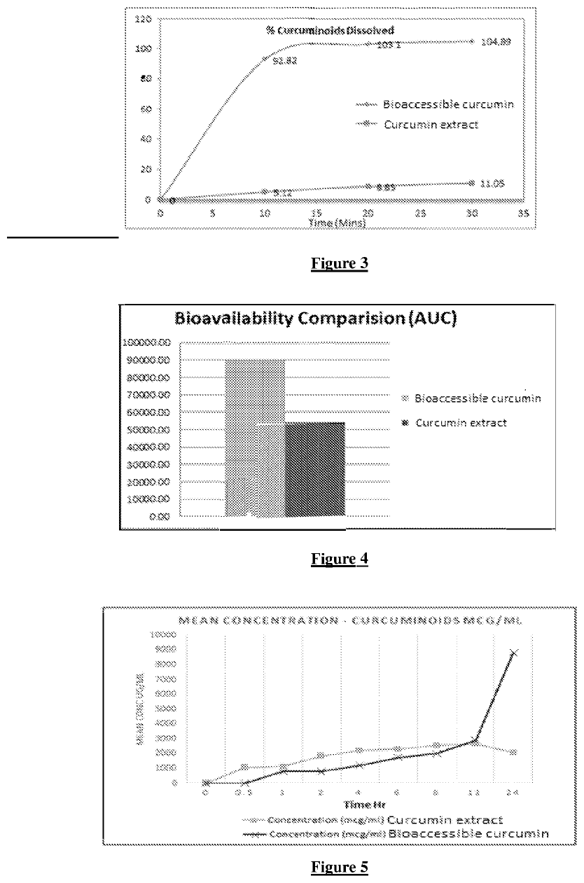

The study results are graphically presented in FIG. 4 and FIG. 5.

Observations: In this study investigational product dose was 500 mg/kg for both products. The Cₘₐₓ of Bioaccessible curcumin of the present invention was observed about 16 times greater than Curcumin 95% Powder. The AUC_{inf} of bioaccessible curcumin of the present invention was observed about 8 times greater than Curcumin powder.

### Example 10

Study was conducted to assess bioaccessible curcumin's, obtained by the present invention, efficacy and safety in treatment of rheumatoid arthritis (RA). The product was administered to the patients with active rheumatoid arthritis for the period of 90 days (3 months) and the efficacy was evaluated in terms of ACR-20, WOMAC Index and Pain VAS using subjective questionnaires.

Study Objective:

To assess the efficacy of bioaccessible curcumin, obtained from present invention, and placebo in active rheumatoid arthritis patients in terms of ACR-20, VAS and WOMAC scale. The secondary objective to assess the safety of bioaccessible curcumin in active rheumatoid arthritis patients.

Study Design:

24 Subjects were recruited for the study. The subjects were asked to take Bioaccessible curcumin 250 mg capsules orally twice per day (one in morning after breakfast and one at night after dinner) and placebo capsules as per randomization sequence. The study/supplement duration was 12 weeks (3 months). The primary and secondary efficacy parameters were ACR-20, WOMAC Index and Pain VAS score and their change from baseline till end of the study were measured. In this study adverse events were observed through; Vitals-Heart Rate, Blood Pressure and Respiratory Rate.

The inclusion criteria for this study were:

a) Patients in the age group of 18-65 years, either male or female

b) Diagnosis of RA according to the revised 2010 American College of Rheumatology (ACR) criteria (with RA functional class of II). Total number of the Swollen Joint Count (SJC) and Tender Joint Count (TJC) should be greater than 8

c) Continuing or recurring pain (i.e., joint pain daily, unless on pain medication)

d) Ability and willingness to participate in all components of the study

e) Patients who able to understand and sign written informed consent.

The exclusion criteria were:

a) Patients to whom one of following applies will be excluded: dysfunction of liver; severe cardiovascular, urinary, hematopoietic, or endocrine system disease; immunodeficiency; uncontrolled infection or active gastrointestinal tract disease; recent vaccination; gravida; women in lactation period or those recently intending to become pregnant; hypersensitivity to study product; treatment with any other disease-modifying anti-rheumatic drug (DMARD) or NSAIDS within 30 days before enrolment; history of alcohol abuse; history of hyperglycemia or motor coordination disorder; or participation in other clinical trials within 3 months before enrolment

b) Patients with any surgical procedure, including bone or joint surgery or synovectomy within 12 weeks prior to screening

c) Patients with significant systemic involvement of secondary RA (including diseases such as vasculitis, pulmonary fibrosis or Felty's syndrome), inflammatory joint disease other than RA (e.g. gout, psoriatic arthritis, seronegative spondyloarthropathy and Lyme disease)

d) Subjects with other systemic autoimmune disorder (e.g. systemic lupus erythematosus, inflammatory bowel disease, scleroderma, inflammatory myopathy, overlap diseases.

The data were assessed by suitable statistical analysis like student's paired and unpaired t-test analyses. Only the results with p≤0.05 were considered statistically significant to rule out the null hypothesis.

Study Results:

A] Change in American College of Rheumatology (ACR-20)

Based on the norms for ACR 20 Improvement, greater than 20% improvement is required in painful joint count, swollen joint count and >20% improvement in 3 of the following five areas: Patient Pain Assessment (PN), Patient Global Assessment (GL), Physician Global Assessment, Patient Self-Assessed Disability (FN) and Acute-Phase Reactant (ESR or CRP). There was 15% improvement achieved in 4 of the ACR-20 criteria in Bioaccessible curcumin (concentration 18%, 36%, 36% and 36%) but in placebo only FN and PN criteria showed the improvement. At the end of the treatment, Bioaccessible curcumin exhibited an improvement of 12% in RA symptoms as assessed by physician's global assessment. Total number of painful joints decreased by 18% and swollen joints decreased by 8% in Bioaccessible curcumin group. Bioaccessible curcumin exhibited an improvement of 20% in function index as per Patient Self-Assessed Disability (FN). It further improved by the end of 90 days treatment to 36%, but in placebo group, an improvement of only 20% was observed. Bioaccessible curcumin showed an improvement of 36% in RA symptoms as assessed by patient's global assessment which was only 22% in placebo group. Thus, apart from remarkable reduction in number of painful and swollen joints, Bioaccessible curcumin, also exhibited an improvement of ≥20% in 4 out of 5 ACR-20 criteria related to subjective assessment by physician or patient.

B] Change in Western Ontario & McMaster Universities Osteoarthritis Index (WOMAC)

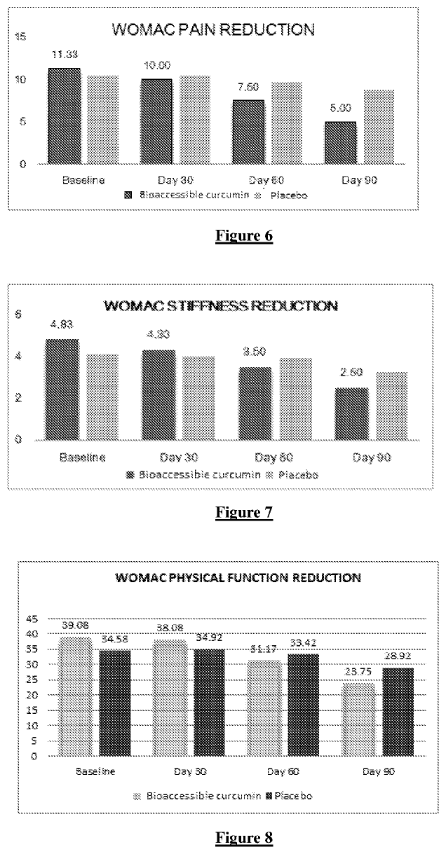

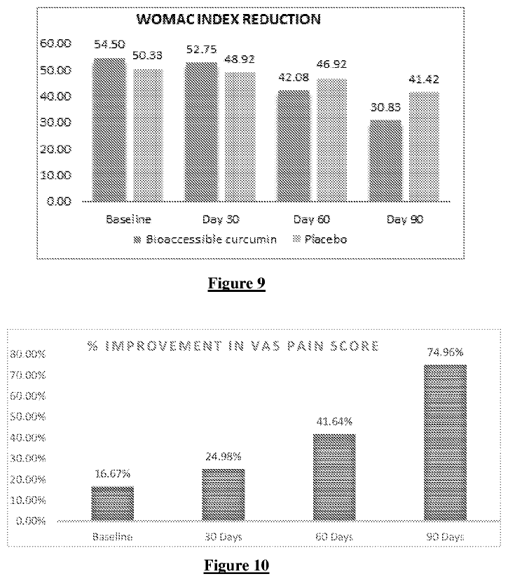

In WOMAC Pain, Bioaccessible curcumin showed statistically significant within and between group p values (FIG. 6). But in WOMAC stiffness only Bioaccessible curcumin showed statistically significant reduction in stiffness intensity from day 30 to day 90 of treatment. Bioaccessible curcumin also showed superiority compared to placebo group as evident from intergroup p values (FIG. 7). For WOMAC physical function within 60 days post Bioaccessible curcumin supplementation, there was significant reduction in physical function difficulty where as it showed only 90 days in the case of placebo. This result indicates that Bioaccessible curcumin has better physical function improvement (FIG. 8). There was substantial reduction in total WOMAC-Index was observed within 60 treatment days but in case of placebo this was only observed during 90 days. Bioaccessible curcumin was also superior compared to placebo group as evident from intergroup p values (FIG. 9).

C] Change in VAS Score

There was significant reduction in Pain (VAS) score in case of Rheumatoid arthritis patients administered with Bioaccessible curcumin capsules. The reduction in VAS within 30 days was 24.98%, reduction in VAS at 60 days was 41.64% and reduction in VAS at 90 days was 74.96%. It was evident from the data that almost 75% subjects benefitted from Bioaccessible curcumin supplementation within 3-month period (FIG. 10).

D] Safety Results

Both the treatments exhibited the vital parameter values within the clinically normal range throughout the study period. Subjects were monitored for Adverse Events (AEs) and Serious Adverse Events (SAEs) and no such incidence occurred throughout the study duration. Bioaccessible curcumin was overall safe and well-tolerated in all subjects when given up to 12 weeks.

Bioaccessible curcumin proved to be safe as well as well tolerated and effective treatment for reducing symptoms of RA with reduction in associated pain and stiffness and improvement in physical function for healthy middle-aged and older RA patients. Moreover, with a minimal daily dose of 500 mg Bioaccessible curcumin (containing 100 mg Curcuminoids), the investigational product showed better efficacy with no adverse effects. It is important to note that very commonly recommended daily dose of Curcumin starts with 1-2 g and based on the clinical outcome Bioaccessible curcumin stands unique as a potent Curcumin branded Ingredient.

From the above clinical data, we see that Bioaccessible curcumin is safe as well as well tolerated and effective treatment for reducing symptoms of rheumatoid arthritis with reduction in associated pain and stiffness and improvement in physical function for healthy middle-aged and older RA patients.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

1. A dispersible composition of lipophilic compounds having enhanced bioaccessibility.

2. The composition of claim 1, wherein the lipophilic compounds are derived from plant and/or synthetic sources.

3. The composition of claim 1, further comprising one or more lipophilic active(s), a hydrophilic carrier, a micelle forming agent, and, optionally, an acidifier.

4. The composition of claim 3, wherein the one or more lipophilic active(s) has a range of 1% to 60%, the hydrophilic carrier has a range of 10% to 90%, the micelle forming agent has a range of 1% to 50%, and the optional acidifier has a range of 0.5% to 10%.

5. The composition of claim 4, wherein the one or more lipophilic active(s) has a range of 5% to 50%, the hydrophilic carrier has a range of 40% to 70%, the micelle forming agent has a range of 1% to 5%, and the optional acidifier has a range of 1% to 5%.

6. The composition of claim 3, wherein the one or more lipophilic active(s) is selected from the group consisting of: curcumin, demethoxycurcumin, bisdemethoxycurcumin, bis-o-demethylcurcumin, tetrahydro curcumin, lutein, zeaxanthin, carotenoids, beta-carotene, boswellic acid, green tea extract, green coffee extract, resveratrol, hypericin, bacosides, xantho compounds, xantho extracts, rhizol, pyrogallol, genistein, wogonin, morin, Silymarin, flavonols such as quercetin, froskolin, kaempferol, flavones such as luteolin and apigenin, hydroxybenzoic acids such as gallic acid, protocatechuic acid, ellagic acid (EA), and vanillic acid, flavanones cardinal aglycones such as naringenin, hesperetin, and eriodictyol, and combinations thereof.

7. The composition of claim 3, wherein the one or more lipophilic active(s) comprises curcumin extract.

8. The composition of claim 7, wherein the composition comprises a concentration of dispersible curcumin of between 5% and 50%.

9. The composition of claim 8, wherein the dispersible curcumin concentration is 5%, 10%, 20%, 40% or 50%.

10. The composition of claim 1, wherein the composition provides bioaccessible curcumin that shows a therapeutic effect at a minimal daily intake of 500 mg, wherein the 500 mg is equivalent to 100 mg of curcuminoids.

11. The composition of claim 3, wherein the hydrophilic carrier is selected from the group consisting of: Carboxymethyl cellulose, methyl cellulose, hydroxyethyl cellulose, Hydroxypropyl cellulose, Hydroxypropyl methyl cellulose, starch, modified starch, gelatin, lactose, mannitol, acacia, carbomer, dextrin, xanthan gum, Arabic gum, maltodextrin, aqueous shellac, liquid glucose, polyvinyl pyrrolidone, polyethylene glycol, glycerol, sucrose, and combinations thereof.

12. The composition of claim 3, wherein the hydrophilic carrier comprises modified starch.

13. The composition of claim 3, wherein the micelle forming agent is selected from the group consisting of: vegetable or edible oils, polysorbates, lecithins, sucrose ester gums, penova ester gums, phopsphotidylcholines, glycerol and derivatives, glyceryl mono stearates, glyceryl distearate, and combinations thereof.

14. The composition of claim 3, wherein the optional acidifier is selected from the group consisting of: citric acid, ascorbic acid, tartaric acid, malic acid, fumaric acid, lactic acid, formic acid, acetic acid, propionic acid, butyric acid, sorbic acid, and combinations thereof.

15. The composition of claim 1, wherein the composition is dispensed in a dosage form suitable for oral delivery.

16. The composition of claim 1, wherein the composition has enhanced bioaccessibility and is prepared by a method comprising:
dissolving the one or more lipophilic active(s) in a solvent or mixture of solvents to form a clear solution;
dissolving the hydrophilic carrier in water;
adding the lipophilic active phase in hydrophilic carrier phase under homogenization to form a micelle;
dispersing the micelle in a micelle forming agent that optionally includes an acidifier; and
removing the solvent or mixture of solves and drying to obtain a powder.

17. The composition of claim 10, wherein the bioaccessible curcumin has a function selected from the group consisting of: anti-inflammatory, anti-cancer, anti-microbial, neuroprotective, prophylactic treatment of rheumatoid arthritis, prophylactic treatment of osteoarthritis, prophylactic treatment of liver cirrhosis, prophylactic treatment of asthma, and combinations thereof.

18. The composition of claim 10, wherein the bioaccessible curcumin helps in elevating testosterone level, promotes weight loss by lipolysis, and has a potential antioxidant effect.
